(19) United States
(12) Patent Application Publication
(10) Pub. No.: US 2011/0224518 A1
(43) Pub. Date: Sep. 15, 2011

(54) HANDHELD APPARATUS TO DETERMINE THE VIABILITY OF A BIOLOGICAL TISSUE

(76) Inventors: Jaafar Tindi, Bronx, NY (US); Farah Khan, Shrewsbury, MA (US); Christopher D. Hue, Flushing, NY (US); Chinedu Chukuigwe, New York, NY (US); Saumya Banerjee, Flushing, NY (US); Rohan Shah, Edison, NY (US); Alina Levchuck, Edgewater, NJ (US); Marom Bikson, Brooklyn, NY (US); Luis Cardoso, New York, NY (US); Prasad Adusumilli, New York, NY (US); Nabil Rizk, Mamaroneck, NY (US); Luis Carlos Oliveira, Newark, NJ (US); Varun Bansai, Maspeth, NY (US)

(21) Appl. No.: 13/054,862

(22) PCT Filed: Jul. 22, 2009
(86) PCT No.: PCT/US09/51424

§ 371 Date: May 16, 2011

Related U.S. Application Data
(60) Provisional application No. 61/082,658, filed on Jul. 22, 2008.
(60) Provisional application No. 61/198,314, filed on Nov. 5, 2008.

(51) Int. Cl. A61B 5/1459; A61B 6/00

(52) U.S. Cl. 600/323; 600/407; 600/310

(57) ABSTRACT

The present invention provides for a handheld apparatus for in vivo examination of the viability of a biological tissue.

### FIELD OF THE INVENTION

The present invention is directed to a handheld apparatus capable of determining the viability of a biological tissue, such as oxygen level, especially during surgical procedures.

### BACKGROUND OF THE INVENTION

There have been attempts in the past to indirectly analyze the blood supply changes in a biological tissue by employing non-invasive techniques such as laser Doppler flowmetry, surface oximetry-capnography, photoplethysmography and mucosal pH monitoring. These studies involved taking measurements at particular times during surgery, thereby giving a direct or indirect evaluation of perfusion and/or oxygen supply. However, there has been no disclosure that we are aware of that describes a handheld device that is easy for the surgeon to use and is able to provide reliable data on the viability of a biological tissue generally, and more specifically the blood supply changes in that tissue.

### SUMMARY OF THE INVENTION

The present invention provides the specifications, fabrication, operation, performance of a handheld apparatus for the in vivo examination of the viability of a biological tissue.

The viability of the tissue that is determined by the apparatus of the present invention can be oxygen level, oxygen saturation, carbon dioxide level, carbon monoxide level, temperature, metabolite level, average blood flow, uniformity of blood flow, density, pulse rate, tissue type, cell type, cell structure, cell death, ion concentration, hemorrhage, metabolic rate, electrical activity, electrical resistance, pH, neuro-modulator concentration, and/or neurotransmitter concentration or any combination of the above or any physiological parameter that can be inferred by measuring the above. These detection methods are known by those skilled in the art. For example, certain methods for determining the viability of a biological tissue are disclosed in U.S. Pat. No. 6,084,611; U.S. Pat. No. 4,509,522; U.S. Pat. No. 5,615,672; U.S. Pat. No. 4,223,680; Z. Zhong et al., Optics Express, Vol. 16, Issue 17, pages 12746 to 12756; and J. de Vries et al., Medical and Biological Engineering and Computing, Vol. 31, Number 4, July, 1993. The disclosures of these are incorporated in their entirety by reference.

As one embodiment of the present invention, the present invention provides an in-house pulse oximeter to be used for the intraoperative measurement of oxygen saturation (SpO2) during bowel resections. The device would enable the surgeon to attain better results during small bowel resection by providing real-time information of localized blood oxygen saturation. By using such a device, the surgeon would perform an anastomosis at a site with more favorable blood oxygen saturation as a preventative measure to postoperative anastomotic leakage.

The clinical signs of an anastomotic leak include the presence of hematomas or seromas at the neck wound (site of operation), localized inflammation and expulsion of air or saliva. Estimates obtained from post-operative care indicate that 20% of all patients that undertook bowel resections suffer from anastomotic leaks. Literature reports indicate that the mortality rate due to such leaks typically lie within 10% to 15%. Furthermore, anastomotic leaks have been associated with increased local recurrence of the disease and reduced survival rate after cancer surgery. (Lerut et al., 2002)

Anastomosis leaks of the gastro-intestinal tract are caused due to poor blood supply to the anastomosed ends, perforation in the tissues, or inadequate re-attachment of the relevant organs. For instance, during esophageal surgery, the stomach pouch is stretched into a tubular conformation and attached to the esophagus near the neck. Surgeons remove the blood vessels supplying the stomach, except for the right gastroepiploic artery and vein (Lieberman et al., 1992). When the stomach is stretched, it exerts strain on the lone artery and increases the risk of a possible leak due to decreased blood supply. The decreased blood supply in turn reduces the oxygen supply to the anastomosis of the gastric conduit. The device of the present invention allows the surgeon to identify the site most likely to heal after surgery, monitor the viability of that site during surgical preparation and at time of surgery.

The following discusses certain aspects of the handheld apparatus of the present invention.

A key aspect to obtaining good readings relates to the positioning the sensor on the target tissue in a convenient manner that allows the user to simultaneously hold the sensor on the target tissue while looking at the display on the device (and perhaps conducting yet another simultaneous procedure). The first aspect of the invention relates to the articulation (or use of joints) in the design of the handheld apparatus. The device can be composed of several distinct components which are typically connected in series with joint members. These components are from most distal (at the tissue) to most proximal (nearest the user):
1) The sensor assembly comprising at least one optical emitter and at least one optical detector;
2) The sensor holder, a relatively small enclosure containing at least the optical detector(s). The holder can be rectangular with one open side for the detector(s).
3) The shaft. The wiring from the sensor assembly to the main processor runs through the distal (nearer to the holder) and proximal shaft (further from the holder). The distal and proximal shaft may be cylindrical.
4) The device head which contains a processor which is in communication with the sensor assembly to provide data on the viability of the target tissue. The device head may be square.
5) The power unit to power the optical emitter, optical detector and processor; and
wherein at least the holder and the distal portion of the shaft are connected by a joint member.

The joint member(s) of the device between any of these components are designed in a manner that allows for the device to be configured with an angle between these components. Additional elements can be added to the above components, with or without additional joints, that would serve the same function but allow for additional functions.

It should be obvious that it is possible to mechanically combine certain elements, add elements, or re-arrange elements without changing the “jist” of the invention. For example, one could shape the device head so as to make the head has a similar form factor to the shaft or to fuse one end of the head with the shaft. As another example, one could add power units or additional processors to the shaft or sensor holder. As another combine, one could integrate the hand-held device with another sensor or surgical instrument. As another example, one could modify the device to be compatible with another instrument for example making the exterior resistant to certain chemicals or heating or making the device out of “MRI-compatible” materials

The angle between the primary axis of one component and the primary axis of another component may be acute or obtuse. Each component may have its dimension restricted to a predetermined limit. It is usually desirable that the components be as small as possible, except the monitor where it may be desirable to have the data visually displayed as large as possible to make it easier to read. Generally, the device head is a bit larger to accommodate the main processor and the power unit. The shafts may be rather narrow as they only accommodate connector cables. There is a desire to make the sensor holder as small as possible to allow the sensor assembly to focus the detection area on a small portion of the biological tissue—however, the optical emitter(s) and optical detector(s) themselves, and the minimal distance between them, can only be reduced “so much” without degrading signal.

The overall design of the joint members is to allow the user to target the tissue of interest with the sensor assembly, while holding the device in a comfortable manner, and looking at the display.

The joint member may be flexible. For example in the case of the sensor holder, it can be connected to the distal shaft in a manner similar to a shaving blade—allowing it to naturally bend or pivot with the tissue. Preferably, the open side of the sensor holder should be parallel with the surface of the tissue of interest. Alternatively, the distance between the sensor elements and the tissue can be minimized through this rotation. Further, because the sensor holder walls (the side that are not open) may block “ambient” disruptive light—this rotation results in the tissue of interest being optimally covered by the sensor holder, minimizing invasion of ambient light.

Rubber bands and springs and other mechanisms may be incorporated into the flexible joint members to have them take a default position when not pressed by the user. Other mechanical components can be used to provide some resistance to motion, prevent specific motions (for example beyond a prescribed range), or link motion in one joint with motion in another joint or mechanical device.

The handheld apparatus of the present invention may further comprise a monitor in communication with the processor to display the data on the viability of the biological tissue. The data on the viability of the biological tissue may be displayed relative to another part of the biological tissue, another biological tissue, or a reference standard. The monitor may display this data visually and/or by sound. The monitor may be attached to the device head, or be separate from the apparatus and in wireless or wired communication with the processor.

Another aspect of the invention is that the data may be depicted in a qualitative or multi-factor display. In a qualitative display, rather than a specific number indicating a specific aspect of tissue quality being shown, a relative reading is displayed. What makes this a relative reading, rather than a specific number, is that this reading is not reference to an absolute scale and is not in any specific units. The monitor may display a measure of the viability of the biological tissue relative to another part of the biological tissue, another biological tissue, an arbitrary value, or a reference standard.

For example, a specific number might be 1 cm, while a relative reading might be a light that turns “greener” with increase size. A relative reading may only be “useful” within a specific subject or tissue, meaning that while one can say “this side of the tissue is greener than that side of the tissue”, one can say what the green means in absolute terms or relate it to another person. The relative reading may be shown in a variety of ways including a color bar, an analog meter, or even a number reading (though this numeric reading is relative).

We also envision a display when multiple values are shown at once. This is at least one “primary” relative (e.g. green) or “primary” absolute (e.g. temperature) value in combination with one or more “additional” distinct relative or absolute values. For example we can show the “green” indicator with a specific and absolute temperature reading. The “additional” values shown may provide distinct information on the tissue, or may provide information on the device performance, or may provide information on the device performance relative to the tissue. For example, the “additional” value may indicate the distance of the sensor from the tissue, or if contact as been made, or what the quality or pressure of that contact was. Now the key with the multi-factor display is that the “primary” and “additional” features are that they are read together by the operator. They together provide useful information to the operator. For example, “distance from tissue” in combination “green indicator” may provide information that neither quantity on its own provides—if the distance is too large, than the green indicator may be providing reliable information and the device should be moved. As another example, a primary value “tissue temperature” in combination with relative indicator of “level of noise”, may let the clinician know how much he can “trust” the primary value and if they should “trust” the temperature reading and if they should adjust how they are holding the device.

Another aspect of the invention is related to SECONDARY sensors and signal processing (a system) that provides information about the quality of the contact with the tissue. This information is then used to provide feedback the operator and how much they can trust the PRIMARY sensor reading. The primary sensor is the one being used to detect the physiological signal of interest.

The secondary sensor can be used to detect the position of the holder relative to the tissue, the position of the optical emitter relative to the tissue, the amount of ambient light the optical detector or the biological tissue is exposed to, or a characteristic of the tissue or the apparatus. The data from the secondary sensor can then be displayed by a monitor.

For example, the secondary detector is capable of detecting the position of the holder, the distance the holder is away from the surface of the tissue, the contact between a portion of the holder and the tissue, contact between a portion of the holder and fluid near the tissue, or the pressure between the holder and the surface of the tissue.

The characteristic of the tissue the secondary sensor may detect may be the temperature of the surface of the biological tissue, pulse rate, regularity of pulse rate, blood flow, changes in blood flow, uniformity of blood flow, disruption of blood flow, tissue oxygenation, spatial distortion, compression of blood vessels or tissue, damage of blood vessels or tissue, tissue strain, tissue stress, tissue bending, tissue deflection, tissue stretching, tissue hemorrhage, tissue compression, moisture, or density.

The secondary sensor may also detect the configuration of the apparatus, such as the angle between the holder and the distal portion of the shaft.

A key aspect of this embodiment of the invention is that there is an additional or modified sensor that is being used. This information may also be used by the operator to adjust things such as the position of the sensor or the ambient conditions. The secondary sensor reading may be absolute or relative and may be visual or audio. For example a beeping noise may indicate if distance from the sensor to the tissue. Examples of secondary sensors include sensors for distance, contact, pressure, and temperature. Secondary sensors may also help protect the tissue (e.g. too much pressure).

We can describe a range of potential secondary sensor classes including those using electricity/resistance, deflection/mechanical contact, moisture, temperature, and or light, accelerometer, localization relative to a external reference or surface.

It should be obvious to anyone skilled in the art that more than one secondary sensory can be used under specific circumstances to further assist operation or that a secondary sensory sensitive to more than one quality may be used.

A further aspect of the invention is related to additional elements that help position the sensor in a way that increases signal quality and/or protects the tissue. We already mentioned the pivoting joint to allow the holder to align with the biological tissue.

The handheld apparatus of the present invention may also have a cuff around the opening of the holder to at least partially block the ambient light from the optical detector. Preferably, the cuff is compliant. For example, the cuff may comprise a foam, a gel, rubber or spring. Alternatively, the cuff may be attached to the sensor head or shaft via a compliant material. Alternatively the cuff made is made of materials that can move relative to each other, for example like a fan with overlapping blades, resulting in effective compliance. Alternatively, the cuff made of materials that break under specific mechanical conditions.

Alternatively or in addition to the cuff, the handheld apparatus may have at least the opening of the holder covered by a casing to prevent contact of at least the optical detector with the biological tissue, wherein the casing is substantially transparent to the light from the biological tissue. It would also be preferable if the casing is substantially transparent to the light emitted by the optical emitter.

A casing may cover the opening of the holder, or the holder and the shaft, or the holder and device head, or the entire device. The casing is removable, may be disposable, and does not interfere with the function of the sensor, the reading of the display, or any of its functions.

It should be obvious to anyone skilled in the art that though the cuff and casing have distinct functions, they may share certain features or be integrated into a single device or series of devices. For example, the cuff may be disposable. Or the cover may be attached to the cuff.

In order to remove fluid or other material that may compromise the detection of the viability of the biological tissue, an element may be added in or near the holder to deliver a solution to wash the holder and/or remove the fluid or other material from the holder and/or condition the distal component of the device or tissue. A heating or cooling element may also be added in or near the holder to heat or cool the tissue to a predetermined temperature or condition the interface between the device and the tissue.

### BRIEF DESCRIPTION OF THE DRAWINGS

FIG. 1 depicts in schematic form the manner a user can adjust the handheld apparatus of the present invention to obtain an accurate measure of the viability of a biological tissue using primary and secondary sensors.

FIG. 2 depicts in schematic form how the signals from the primary and secondary sensors are processed by the processor(s) to provide quantitative and/or qualitative data as the viability of a biological tissue considering the condition of the tissue, such as temperature, and the environment affecting the tissue, such as surface conditions, presence of fluids, and degree of compression by the handheld device.

FIG. 3 shows a monitor that is connected to a body such as the device head through two hinge joints that allow for at least two range of motion for the monitor.

FIG. 4 shows the holder of the sensor assembly in a form of a movable tip wherein a slot in the distal portion of the shaft allows the holder to change its angle with the shaft.

FIG. 5 shows an embodiment of the handheld apparatus where a monitor is attached to the device head, which in turn is attached to the shaft.

FIG. 6 shows another embodiment of the handheld apparatus where the shaft is configured in two separate parts connected by a joint member, and the holder is connected to the shaft by another joint member.

FIG. 7 shows hand-held pulse oximeter sensor utilizing reflectance oximetry.

FIG. 8 shows sensor head with photo-emitter and detector.

FIG. 9 shows the prototype casing, sensor head, and area of application on a resection surgery.

FIG. 10 shows a block diagram explaining the step processes involved in the intra-operative pulse oximeter signal acquisition, analysis, and digital display

FIG. 11 shows a circuit schematic of an Oxygen Saturation measurement device

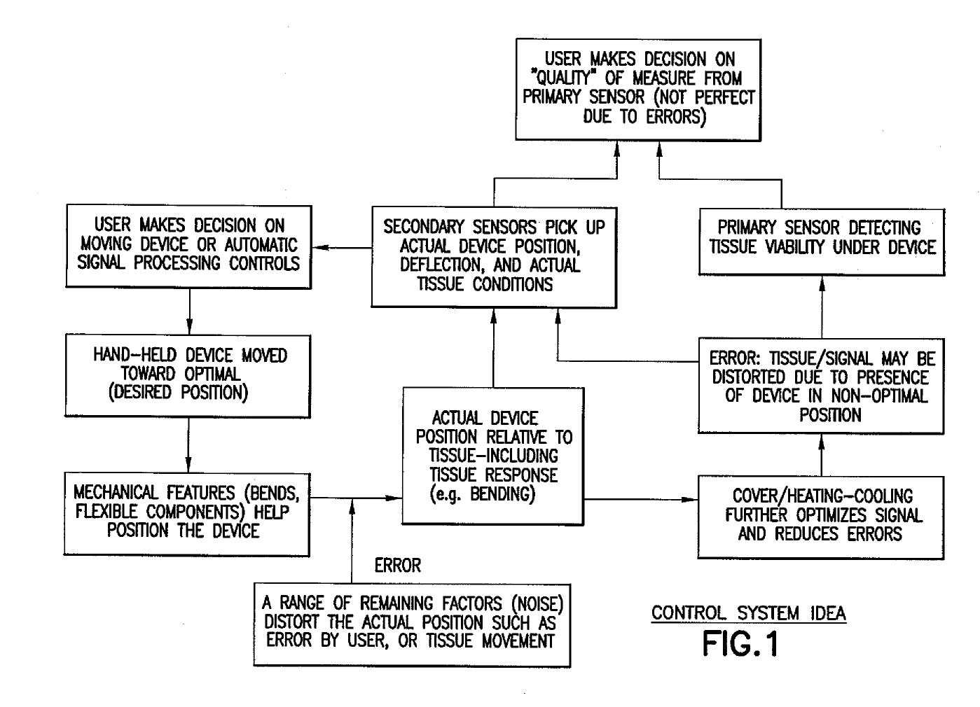

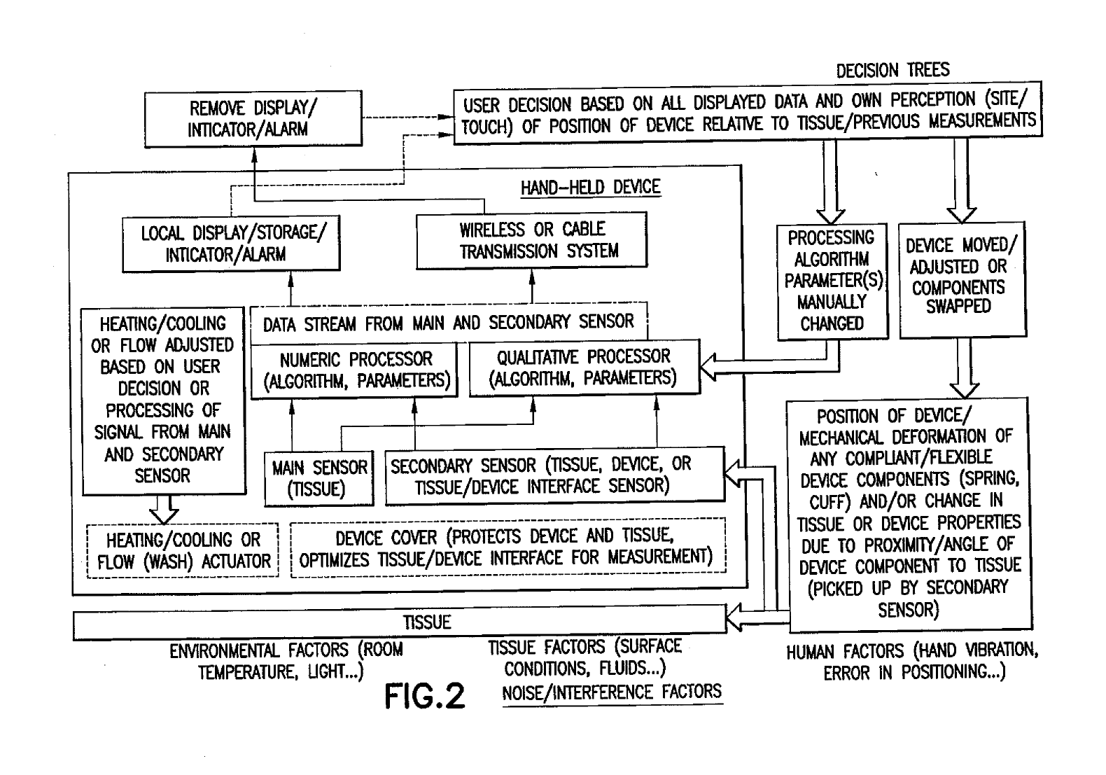

### DETAILED DESCRIPTION OF THE PRESENTLY PREFERRED EMBODIMENTS

Examples of the joint members between the components of the handheld apparatus of the present invention are disclosed below.

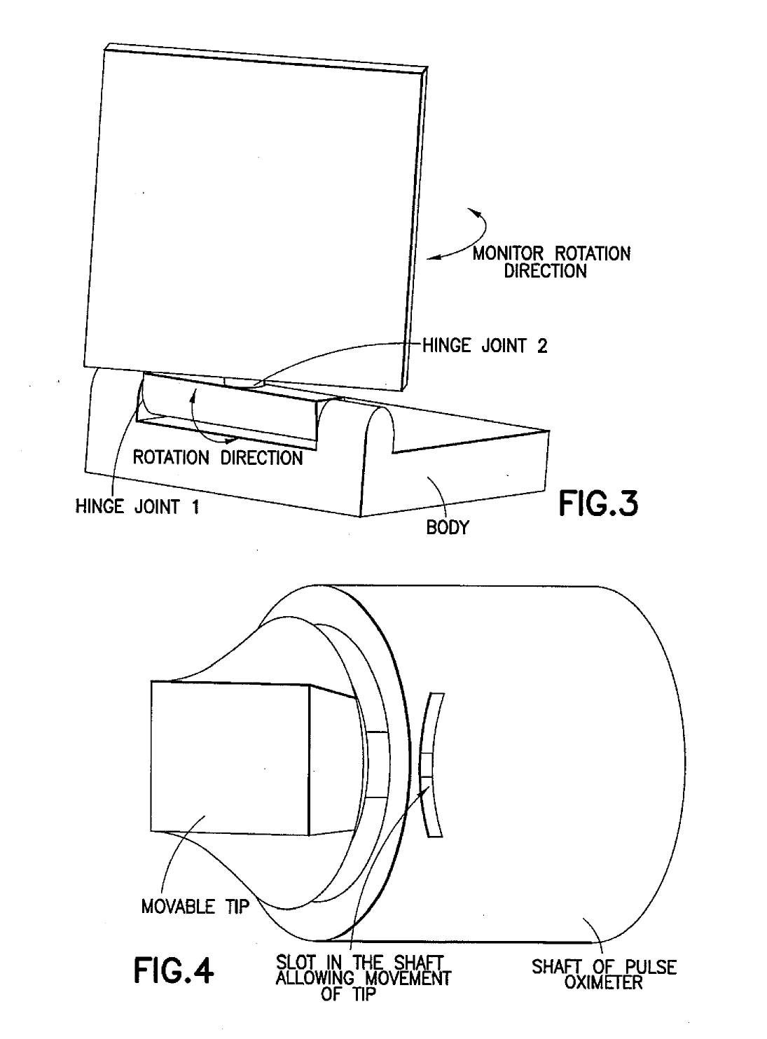

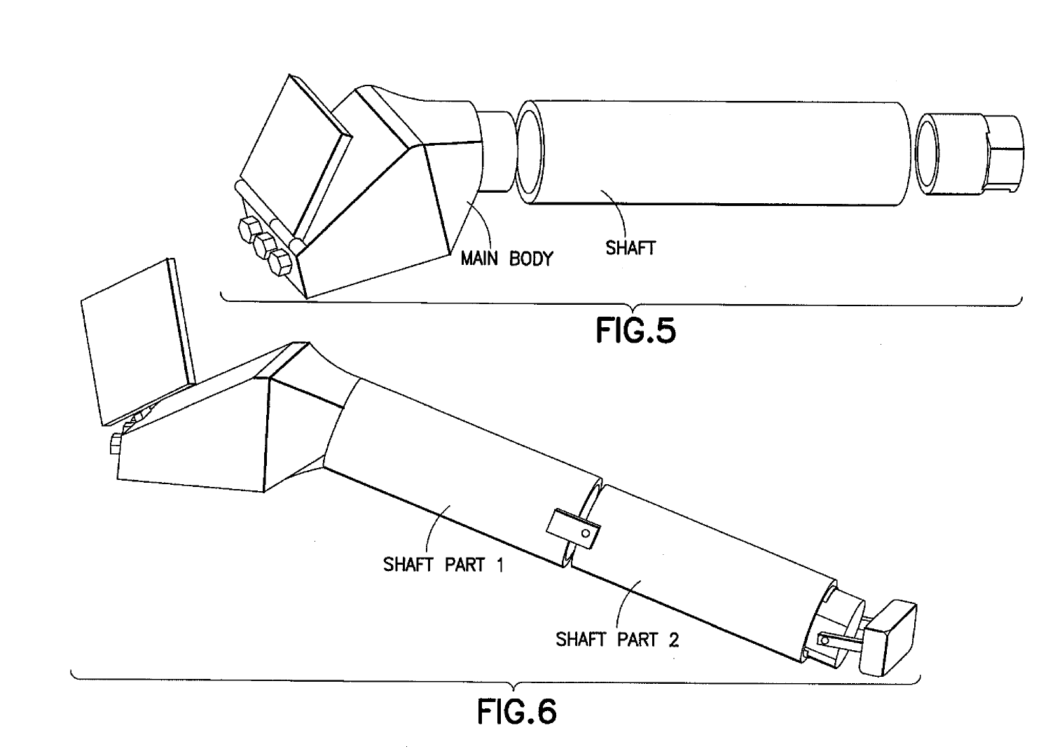

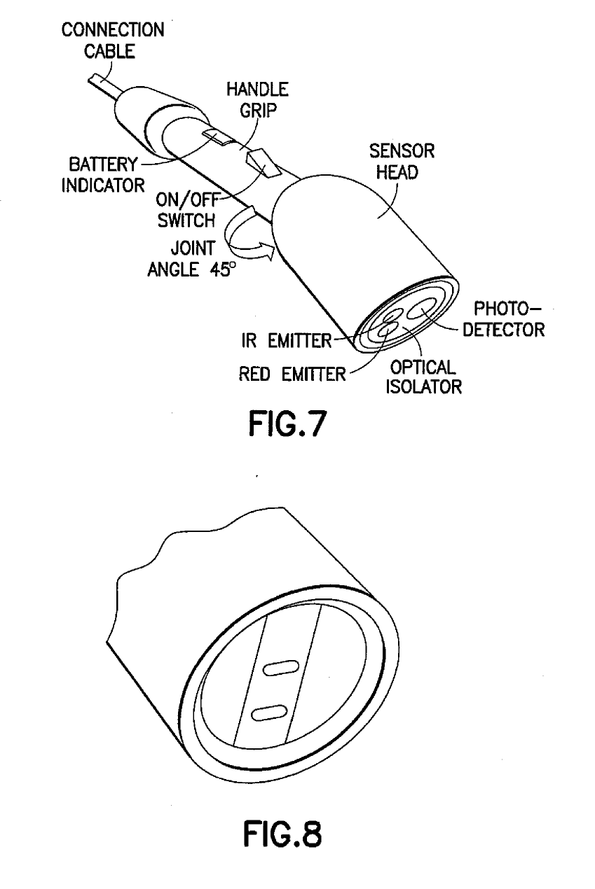

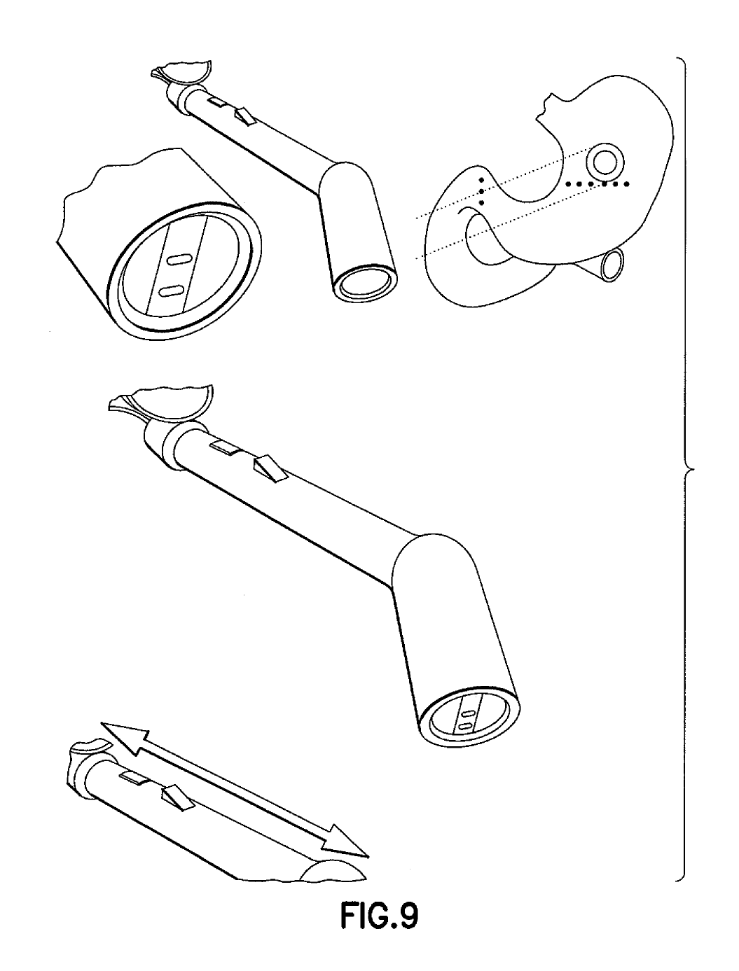

Hinge joint or a combination of hinge joints are examples of joint members. One example is shown in FIG. 3.

There are 2 HINGE joint between Monitor and pulse oximeter Body. Both of them together allow full rotation of monitor in any direction.

Ball and socket joint is another example of a joint member that allows for a full range of movement.

A pivot joint is another example of a joint member that allows movement in two dimensions. An example of such a pivot joint is shown in FIG. 4.

Various forms and combination of the above joint members may be employed in the handheld apparatus of the present invention. Other joint members known by those skilled in the art can be used in the present invention.

### Sensor Assembly

In one particular embodiment, the sensor assembly is configured to measure SpO2, CO and CO2.

The handheld apparatus in one embodiment of the present invention may have two separate modes of operation.

In mode A, device receives signal related to SpO2, CO and CO2 from the sensor. A minimal frequency of 1 KHz for sampling is established. The microcontroller receives respective separate sample values from an internal memory following an algorithm to calculate SpO2 percentage based on SpO2, HbCO and CO2 information. In mode B, user can select device to measuring SPO2, HbCO or CO2 separately.

Depending on the needs of the user, various sensor assemblies with different capabilities may be detached from the holder so that another sensor assembly may be substituted into the holder.

### Measurement of SpO2

The handheld apparatus measures SpO2 by the following method: SpO2=HbO2/(HbO2+Hb)×100%, where SpO2 is the saturation percentage of oxygen in the blood, so called O2 concentration in the blood; it is defined by the percentage of oxyhemoglobin (HbO2) in the total hemoglobin of the arterial blood. Hb are those hemoglobins which release oxygen.

Due to that HbO2 and Hb have different absorption character in the spectrum range from red to infrared light, the wavelength can be adjusted from 600 nm to 1000 nm in order to determine the saturation percentage of oxygen in the small blood vessels.

Specifications:
a—display mode: OLED Display
b—power supply: 2×1.5 V (AAA size) alkaline battery
c—operating current ≈50 mA
d—SpO2 display: 35%-99%
   CO display: 0.1-90%
   CO2 display: 0.1-99%
e—Pulse rate display: 25 bpm-250 bpm
f—Accuracy: SpO2: 75-99%+−2%
Pulse rate: +−2 bpm or +−2%
g—dimension: . . . (Varun)
h—net weight: . . . (Varun)
i—electro-Magnetic compatibility: Group I, Class B
j—The performance under low perfusion condition:
   The accuracy of SpO2 and PR measurement still meet the precision described above when the modulation amplitude is as low as 0.6%

### Measurement of Carbon Monoxide

The handheld apparatus measures carboxyhemoglobin (HbCO) in a tissue using light transmission at frequencies of 548 nm, 810 nm and 950 nm.

### Measurement of Carbon Dioxide

The handheld apparatus uses Infra-red spectrographs method of measurement of CO₂. The wavelength of IR rays exceeds 1.0 milli micron while the visible spectrum is between 0.4 and 0.8 milli microns. The IR rays are absorbed by polyatomic gases (non-elementary gases such as nitrous oxide (N2O), CO₂, and water vapour. Carbon dioxide selectively absorbs specific wavelengths (4.3 milli microns) of IR light. Since the amount of light absorbed is proportional to the concentration of the absorbing molecules, the concentration of a gas can be determined by comparing the measured absorbance with the absorbance of a known standard. The CO₂ concentration measured with the handheld apparatus can be expressed as percentage CO2 (FCO₂), obtained by dividing CO₂ partial pressure by the atmospheric pressure.

### Secondary Sensor

In one embodiment of the present invention, a secondary sensor is used in the handheld apparatus to measure the pressure the holder is applying to the biological tissue, and in combination of the primary sensor that determines oxygen level allow a surgeon to assess the viability of the biological tissue during surgery.

There are few studies demonstrating that the intravascular blood volume can be assessed by the compressibility of a vessel by an ultrasound probe, which can be applicable to the embodiment of the present invention.

For example, when a surgeon is operating on stomach/intestine, at time point 1, before the blood vessels are divided, the tissue oxygenation is 88% at pressure units 30 and on increase of pressure to 38, the tissue oxygenation drops to 60%.

One hour into surgery, at time point 2 (few blood vessels are occluded and divided by now to facilitate pulling up the stomach to the anastomotic area), tissue oxygenation now is 84% at pressure 30 units, but drops to 24% on increase of pressure to 38, indicating that tissue oxygenation is getting compromised as surgery progresses due to division of blood vessels and any further division of blood vessels should be avoided. This will be useful during surgery to differentiate which blood vessels can be compromised and which should be preserved.

### Oximeter Prototype:

### Specification & Technical Information

### Pulse Oximeter Probe Design

Among the other proposed design concepts, the probe design was chosen as the pulse oximeter prototype design which could best satisfy the specific purpose of this project. The prototype oximeter operates based on the principle of reflectance pulse oximetry. Reflectance oximetry is more appropriate here as opposed to transmittance, considering the application of the probe on the biological tissue. Hence, using a non-invasive technique the probe provides blood oxygen saturation measurements from a local tissue point. Moreover, the probe is integrated into a computer display which uses Graphical User Interface programmed using Matlab 7.0 to recognize the signal and implement all the necessary calculations for obtaining the blood oxygen saturation measurements. The prototype casing is customized in house using 3-D printer (Dimension STL) and is made of a polymer material called Styrene Resin (ASTP-400). The probe is manufactured using the proposed biocompatible material (medical grade polypropylene) in order to adapt it for intra-operative use.

TABLE 1
design specifications and performance statistics of the device

| Category | Specification |
|---|---|
| Biocompatibility | ASTP- 400 (Styrene Resin) |
| Diameter (Sensor Head) | <30 mm |
| Device Accuracy (70-100% SpO2) | +/−5% |
| Device Operation Time | >8 hours |
| Temperature Range | 20° C.-40° C. |
| Distance (Receiver − Sensor) | >2 mm |

### Implementing the Commercial Nonin Standalone Sensor

A commercially available Nonin sensor (Nonin Medical, Inc., MN) was integrated into the probe design. The Nonin 8000R sensor operates based on reflectance pulse oximetry to measure blood oxygen saturation and provides with a sensitivity of +1-3 digits. The sensor is fairly easier to implement into our design and is held at the bottom surface of the probe secured by optical isolating material.

### Functional Prototype Process Illustration

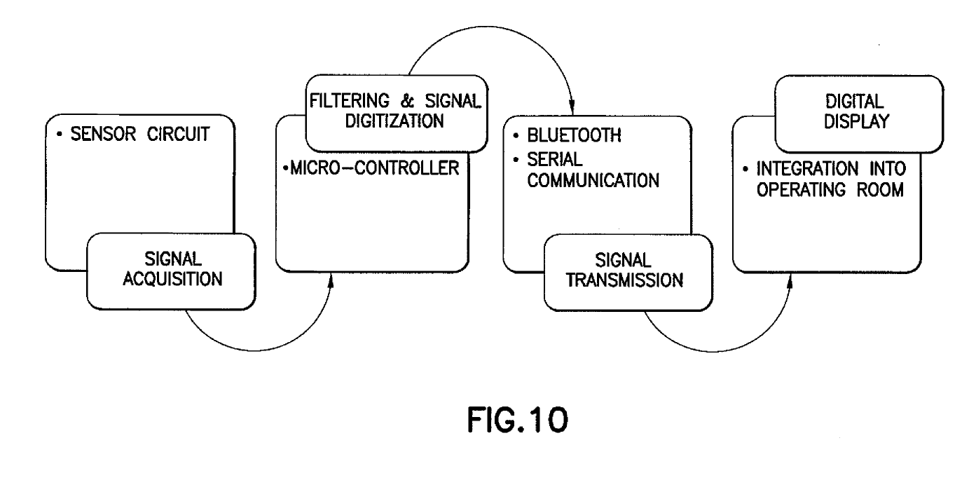

The block diagram (FIG. 10) illustrates the basic functional process of the pulse oximeter device. The pulse oximeter sensor consists of two components where the Red and IR LEDs emit light at two different wavelengths and the photodetector receives an optical signal based on the amount of light absorbed by the tissue. However, the photodetector converts the optical signal to electrical signal which is then acquired by the microcontroller chip which performs the necessary filtering and signal digitization. The digital signal is then transmitted to a computer display (or to the digital display of the operating room) via a serial cable or Bluetooth® communication. The display system then uses a program which has a Graphical User Interface (GUI) to visualize the signal and to obtain the blood oxygen saturation readings using the PPG (photoplethysmogragh) waveforms.

### The Electronic Hardware

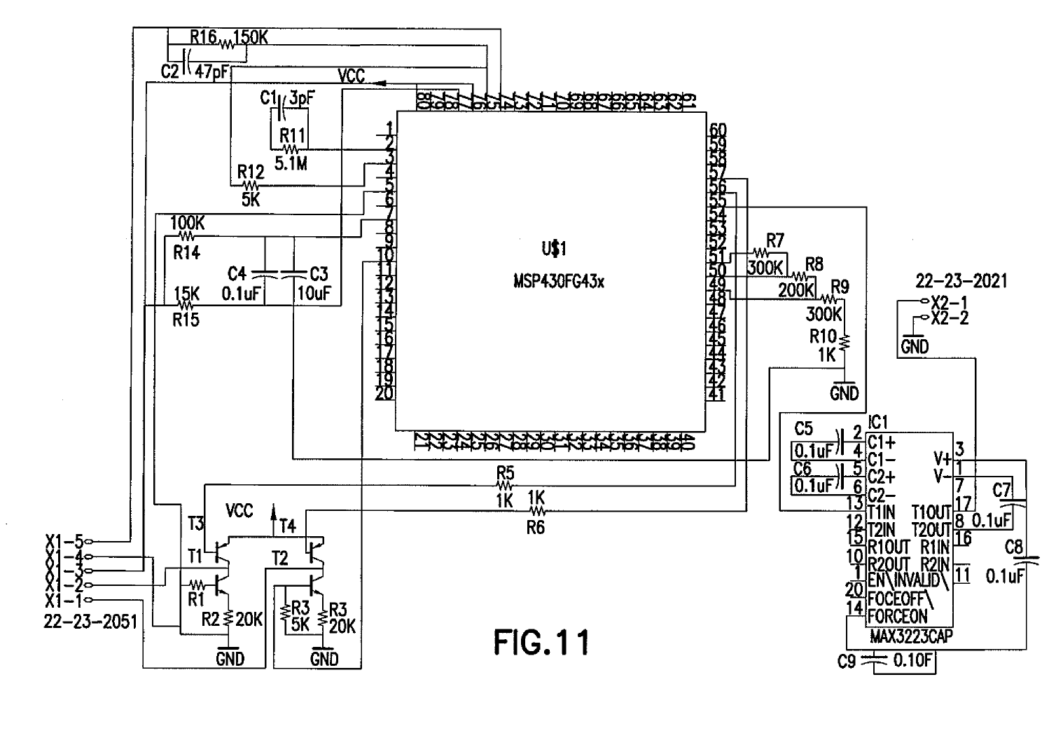

In the following page, FIG. 11 depicts the complete circuit schematic used for the developing a functioning device. The major components include MSP430 Microcontroller (Texas Instruments) and the MAX3223 Transceiver (Maxim Integrated Products, Inc., Sunnyvale, Calif.), discussed in detail further. The signal is then transferred via a serial USB cable (RadioShack, Part #26-183) for further analysis and evaluation using Matlab software.

### MSP 430 Microcontroller Chip (Texas Instruments, Inc., TX)

The microcontroller chip is designed to provide ergonomic signal processing for pulse oximetry and heart-rate detection. The chip employs a principle of light source cycling at 500 Hz to minimize power consumption. The red and infra-red components are then alternately received at the photo diode, which generates a small current. The current is then amplified by the built-in trans-impedance operational amplifier, to produce a strong DC current or about 1V and a small AC current circa 10 mV, which are then sampled and separated by the microcontroller unit. The DC component is a result of the scattering of the light and less oxygen saturated body tissues. The AC component results from the ambient light of 50/60 Hz and the oxygen bearing arteries. Therefore, AC component is the one of greater importance in the pulse oximetry. The DC component is thus extracted and set as an offset for the second operational amplifier, which then amplifies only the AC component. The RMS value is obtained by averaging the square of the signal over a number of heart beat cycles. The real time samples are then transported to the PC via a serial USB cable.

RS-232 Transceiver/Leveler chip MAXIM Integrated Products, CA, model #: MAX-3223E)

The transceiver contains both a receiver and a transmitter. This device is a 3V-powered chip with an automatic shutdown and wakeup feature and enhanced electrostatic discharge protection. The device is capable of saving power without introducing changes to the operating system by enabling the low-power shutdown mode if the connection is altered. The charge pump of the transceiver requires only 0.1 μF capacitors to operate from a +3.0V to +5.5V supply, a range compatible with MSP 430 Microcontroller Chip. The data rate is maintained at 250 kbps. The device also has two non-inverting receiver outputs, which prevent forward biasing.

### Nonin 8000R Sensor (Nonin Medical, Inc., MN)

Nonin sensors are equipped with special Pure-Light technology, which prevents contaminations by the secondary spectrum emissions and eliminates variations from patient to patient. The sensor includes the red LED emitting light at 660 nm (<0.8 mW) and infra-red LED at 910 nm (<1.2 mW). The photodetector receives back-scattered light, giving high accuracy of 70-100% SpO2, with the 3 digit variation.

USB-to-Serial Port Cable (Radioshack Co., TX, Model: 26-183) The USB-to-Serial portal cable facilitates bi-directional communication with the RS-232 Transceiver. The cable connects to a standard (4-pin) USB port and is Intel-compatible (Windows 98, Windows 98SE, Windows ME, Windows 2000, or Windows XP operating systems only). The cable has very low power consumption and is very easy to use.
Component List The following table lists major components used in the prototype assembly.

| Part | Manufacturer |
|---|---|
| MSP 430 Microcontroller Chip | Texas Instruments, |
| | Inc., TX |
| RS-232 Transceiver/Leveler chip | MAXIM Integrated |
| (Model: MAX-3223E) | Products, CA |
| Double Sided Positive Pre- | Altex Electronics, |
| Sensitized PCB (6″ × 9″ × 1/16″) | Ltd., TX |
| Nonin 8000R Sensor | Nonin Medical, |
| | Inc., MN |
| USB-to-Serial Port Cable (Model: | RadioShack Co., TX |
| 26-183) | |
| SPST 25-Amp Illuminated Rocker | RadioShack Co., TX |
| Switch (Model: 275-731) | |

### The Software

Initial considerations of software applications to use led to the choice of LabVIEW, by National Instruments. LabVIEW is powerful data acquisition tool with wide applications in industry. It is fairly straightforward to create acquisition and analysis programs in LabVIEW without the need to write extensive algorithms, as well as create end-user interfaces. Furthermore, it is possible to make stand alone executable programs that can run on computers that do not have the LabVIEW software installed. Unfortunately, LabVIEW requires the use of a National Instruments DAQ card and BNC box to digitize signals and pass them onto a computer. The cost of these devices would greatly increase the cost of our intraoperative pulse oximeter without necessarily enhancing its accuracy and versatility well beyond those of already available (and less expensive) pulse oximeters. In light of these factors, we chose MATLAB, by Mathworks. MATLAB is a technical computing and analysis software based on the C programming language. Unlike in LabVIEW, coding in MATLAB is more involved. Nevertheless, it is possible to create a graphic user interface to enable the end-user to interact with the device without necessarily manipulating the technical algorithm. Further, MATLAB can acquire signals real time from a serial port (e.g., the 9-pin serial connection and USB), or even the National Instruments DAQ card, thus circumventing the need to purchase external acquisition devices as was previously required with LabVIEW. Thus with the choice of MATLAB, there was the flexibility of reading input signals from the probe circuitry via a serial cable (USB or DB9 cable), as well as wirelessly via Bluetooth USB transceiver connected to the computer, all of which were previously not possible with the LabVIEW software. The following describes the implementation of the MATLAB algorithm in signal processing, computation of oxygen saturation and heart rate, and the presentation of these results in a graphic user interface. The actual MATLAB script is presented in the appendix.

### The Matlab Algorithm

### Signal Processing in MATLAB

Currently, the data is read from a serial COM port where the serial cable is recognized. Our MATLAB algorithm configures the appropriate port for data acquisition. The transfer rate is specified as 115200 bauds per second, the buffer size as 2000 and the bytes available for reading as 2000. The data transfer rate is matched with the transmission rate from the microcontroller, which transmits at 115200 bauds per second. Once the port configurations are specified, the port is opened and data read from it as 8 bit unsigned integers. An important note here is that the signal being acquired corresponds to the red and infrared lights flashed into the tissue and picked up by the photodetector. Now, there is no tag on the data being read from the port as to which signal corresponds to which light emission and absorption. MATLAB acquires the signal from a single non-discriminatory channel. As such the algorithm must somehow discriminate and segregate the two signals. This is done by recognizing that the microcontroller sends signals corresponding to the red and infrared emissions alternately. The single channel data acquired by MATLAB from the COM port and stored as a vector of integer values thus contains both of the desired signals in alternating fashion, essentially like the jagged edges of a jacket zipper. Thus to obtain the red and infrared signals respectively (i.e. to open the jacket) the algorithm assigns the odd and even entries to new but shorter vectors corresponding to the red and IR emissions, respectively. With the bytes available to read set as 2000, each of the vectors is 1000 samples long. Since the sampling rate by the microcontroller was 512 Hz, 1000 samples correspond to 1.95 seconds worth of data.

### Filtering

The photoplethysmograph is composed of frequencies in the range of 1-5 Hz. As such it is necessary to filter out frequencies outside of this bandwidth to obtain a clean signal. Our MATLAB algorithm accomplishes this by first obtaining the appropriate filter coefficients corresponding to a third order Butterworth band pass filter with −30 dB attenuation at 1 and 8 Hz cut off frequencies. However, these coefficients are for an IIR filter which by virtue of filtering introduces some phase distortions in the actual signal. To correct for this, the filtfilt( ) function in MATLAB is called as opposed the usual filter( ) function. filtfilt( ) filters the signal forward to remove frequencies in the reject spectrum, and then backwards to cancel any phase distortions that might have been created by forward filtering. In essence, the filtfilt( ) function works like a zero phase filter that operates on the magnitude of the frequency components and not on their phases. Both the separated red and infrared signals are processed in this fashion.

### The Ratio R and Oxygen Saturation

Oxygen saturation is computed based on the differential absorption of the red and infrared wavelengths. In fact, the ratio of red to infrared signal, R, is proportional to the oxygen saturation. However, this dependency is non-linear. As such, current pulse oximeters in the market have an empirically obtained curve that relates R to the oxygen saturation. These empirical curves are used to calibrate pulse oximeters. With the empirical curve replicated from Nonin, the algorithm uses the polyfit( ) function to approximate the Nonin curve with a second order, quadratic, polynomial. Based on this polynomial formula, the algorithm needs to only compute the ratio R from the filtered red and infrared signals and submit that to the polynomial for the calculation of a calibrated oxygen saturation value as a percentage. But even with this calibration, the oxygen saturation output fluctuates. To control for this, oxygen saturation is averaged over 5 values.

### Heart Rate

The arterial pulsations observed in the PPG signal are coincident with the pumping action of the heart. Based on this observation simply counting the train of pulses therefore provides a way of determining heart rate. Although a fairly simple approach, the implementation is not so straightforward. This is largely because the PPG signal becomes unstable due to motion artifacts that dominate when a subject moves. The frequencies of these are within the 1-5 Hz desired bandwidth, so aggressive filtering does not help much either. As a solution, a threshold value of 0.7 times the maximum value of the signal is set in the algorithm, and a counter enumerates the number of times this threshold is exceeded. 0.7 was chosen by observing a stable signal and making the note that other components of the PPG signal were below this threshold, and that only the peak surpassed it. Thus counting the number of times this takes place over the 1.95-second time lapse allows for the calculation of heart rate. Unfortunately, the peak of the PPG is beyond 70% of the maximum value for a given time interval and is not instantaneous. As a result, the threshold counter contains stretches of zeros and ones. What is important from this are the transitions from zeros to ones only, not that the counter gives either a one or zero. Enumerating these transitions outputs the number of peaks, which corresponds to the number of beats in 1.95 seconds. The number of beats divided by the time interval provides the heart rate in Hz. To convert to beats per minute, the outcome is multiplied by 60 seconds. Although this is the current method used, it is obvious that this algorithm suffers whenever motion dominates and the 70% threshold is passed by noise signals, resulting in a higher than normal heart rate. There are also instances where successive PPG peaks depreciate in amplitude and so while the peaks are visibly present and can be discriminated from the noise and other components of the signal, they do not reach the threshold. Consequently, a lower heart rate than normal is registered. A simple correction for this is heart rate averaging. The algorithm achieves this by computing the mean of previous five values. This is similar to the averaging of the oxygen saturation values previously described.

### The Graphic User Interface (GUI)

The purpose of the intraoperative oximeter is not only the measurement of oxygen saturation but also the presentation of the results in a user-friendly, easy to comprehend format. The capacity to design a stand-a-lone GUI was therefore one of the motivations for using MATLAB. The simplicity of a GUI requires no prior training and ensures usability with no technical understanding of the algorithm previously described, or of the code that enables the functionality of the GUI itself. Here we present the graphic user interface with some explanations to its key functions.

### Figure Legend and Axes

Most pulse oximeters only display oxygen saturation as a percent quantity without conveying any information about the PPG signal at all. The graphic user interface presents the red and infrared signals respectively in addition to the standard oxygen saturation and heart rate. Similar to the analysis of the electrocardiogram, an examination of these signals provides some information regarding a patient's respiration. The y-axis gives the scaled and offset amplitude of these signals. The actual values are not important and thus intentionally not shown. The x-axis is the time axis in seconds. At most, only two seconds of data will be shown and updated every two seconds.

### Patient Name *

The option to enter a patient's name is not necessary for the functionality of the GUI. However, it does ensure that a recording session will be associated with a particular patient and not confused with another patient's data once the recording session is complete. Date * The date entry option is also for filing, in case it becomes important to determine when a specific recording was done. *More entry options may be added later upon request, and as deemed necessary, such as the OR used for the recording, the time the recording was initiated, etc. None of these however, have any bearing on the actual signal obtained or the oxygen saturation. Start The start button when clicked activates the algorithm to continuously acquire the signal from the probe via the serial port connection, or wirelessly via Bluetooth (once Bluetooth transmission is enabled). It takes two seconds for the signal to be displayed on the GUI once the start button is clicked. Should it take longer than two seconds, the probe may be turned off or the serial cable may not be properly connected to the computer. Stop This option terminates continuous acquisition and freezes the current display on the axes. The device may be powered on, and transmitting signals to the computer, but the algorithm has been stopped to prevent further signal acquisition, processing and display. To turn off the device and save on battery life, as when the device is no longer being used, the switch on the probe should be turned off as well. Save File Clicking on the Save File button opens up a user interface for saving files, It requests the name of the file and the directory where the data should be saved. Save File stores the patient's name and date options if previously provided, along with the red and infrared signals, the heart rate, oxygen saturation and time axis values. The algorithm stores all this information in a MATLAB data structure (.mat) and can only be accessed by MATLAB at this time. The Save File button should be clicked after clicking Stop. Doing so ensures that all the data since the Start button was initiated is saved, not just the data shown on the GUI.

Print File The Print File button functions in the same way as Save File in that the all the data since the Start button was initiated till the Stop button is clicked is printed. Print File opens a user interface printer dialogue that allows the user to choose the appropriate printer settings prior to printing the file. An important note is that the file being printed is not the GUI itself. However, it is the accumulated data re-plotted in MATLAB with a longer time axis. This is because more data is plotted here than on the GUI (which only displays at most 2 seconds of data). Not all of the data is printed on a single sheet, but stretched over several sheets to allow the discrimination of individual PPG peaks and components. Simply plotting on a single set of axis after acquiring data for about 1 hour would result in a print out with very small peaks squished together so close as to not allow any post recording examination and analysis of the PPG signal.

### Device Operation

Turning on the device and opening associated software files The on/off switch can be found in the rear end of the device probe. Once switched on an on/off LED indicator will light up. The corresponding graphical interface Matlab software should be loaded before the intra-operative pulse oximeter probe is used. The start button should be pressed to run the device continuously while obtaining oxygen saturation readings.

### Intra-Operative Pulse Oximeter Sensor

The head of the probe (containing the Nonin 8000R) sensor is the only component of the device intended to come into contact with the patient. The full surface area of the sensor head (30×30 mm) must be applied to the anastomosed tissue with sufficient pressure (between 4 and 40 kPa) in order to obtain a reliable SpO2 reading.

For the user (surgeon) of the pulse oximeter probe, the shaft should be held firmly in hand. The above figure outlines the shaft handle of the device. While in used the user may hold the device in any comfortable position so that the following application of device to tissue is achieved. After successful contact with the patient, the device must be firmly held in place without movement for seconds. This will allow enough time for the graphical patient information display to be updated with the corresponding SpO2 measurement. The procedure must be followed for every oxygen saturation measurement made thereafter.

### Testing Methods

Comparison with Standalone Pulse Oximeters

The performance testing of our device was conducted and it was rated in comparison to two existing commercial finger pulse oximeters. The Nonin Onyx 9500 is an oximeter that utilizes transmission oximetry to calculate arterial oxygen saturation and also displays the heart rate of the user. Transmission oximetry has a slight advantage over reflectance oximeters due to the positioning of the photo-emitter and detector. Evaluating the performance of the present reflectance oximeter to this device will provide substantial evidence on the accuracy of the prototype. The other oximeter is a SPO 5500 finger reflectance oximeter. The SPO5500 measures blood oxygen saturation (% SpO2) and pulse rate using a patented method of reflective hemoglobin color measurement that does not require light to shine though the finger so it works perfectly even if wearing opaque nail varnish. It provides a good reference point for measuring the accuracy of our device since it utilizes reflectance oximetry. Once the device passes all required safety tests and regulations, the performance assessment is perforated in vivo, via animal testing.

### Gold Standards in Pulse Oximetry

Blood Gas Analysis: An arterial blood gas (also called “ABG'S”) is a blood test that is performed specifically on arterial blood, to determine the concentrations of carbon dioxide, oxygen and bicarbonate, as well as the pH of the blood. Its main use is in pulmonology, to determine gas exchange levels in the blood related to lung function, but it is also used in nephrology, and used to evaluate metabolic disorders such as acidosis and alkalosis. As its name implies, the sample is taken from an artery, which is more uncomfortable and difficult; moreover being an unfeasible approach for local tissue intra-operative oxygen saturation monitoring.

Carbon Monoxide Oximetry (CO-Oximetry): A CO-oximeter is a device for detecting hypoxia, a medical condition relating to oxygen deficiency at tissue level. It is an enhanced version of a pulse oximeter operating with more than two wavelengths of light. The device measures absorption at several wavelengths to distinguish oxyhemoglobin from carboxyhemoglobin and determine the oxyhemoglobin saturation: the percentage of oxygenated hemoglobin (Hb) compared to the total amount of Hb, including carboxy-Hb, met-Hb, oxy-Hb, and reduced Hb. When a patient presents with carbon monoxide poisoning, the CO-oximeter will detect this Hb and will report the oxyhemoglobin saturation as markedly reduced. This test is non-invasive and provides the most accurate representation of oxygen saturation in the blood, however, the instrument (portable/table top) costs between USD1200-6500; which makes it unfeasible as a reference for testing the accuracy of the reflectance oximeter.

### Physiological Testing

pH Testing: The application of the device is in the region of the gastrointestinal tract, where once the resection has been made the open end of the stomach presents a highly acidic environment (pH 1-4). Moreover, the open thoracic cavity is flushed in bodily fluids (mainly water, blood) which have a range between pH 7-7.4. The intra-operative oximeter has to function within this region without being affected by such a large variation in pH. The acidic environment should not affect the polypropylene coating that protects the sensor and the electronic components of the device from the body fluids. Not only does this interfere with sterilization requirements, but also the overall functioning and reliability of the device itself. To test the consistency of the device under such extreme conditions, the prototype will be immersed in a pH regulated solution simulating the acidic environment. Oxygen saturation readings will be taken before and after immersion to test for any variance in measurement under controlled conditions.
Temperature Testing: The device has to function under regular room temperature as well as inside the human body; particularly the stomach tissue, which has a higher temperature (−37° C.). To assure the device operates under a large range of temperature (18° C.-41° C.), the prototype will be placed in a refrigerator to test for lower temperatures. An incubator will be used to simulate the human body temperature range. Oxygen saturation readings will be taken before and after subjection to temperature changes to identify any significant differences in sensor accuracy.

### Operational Performance Testing

The esophageal resection surgery is an extensive procedure that generally lasts from 4-5 hours. During this period, several incisions and adjustments are made to the thoracic cavity prior to accomplishing the resection. The use of the oximeter is after the surgeons cut off blood supply from three arteries and veins supplying the stomach except the right gastroepiploic artery and vein. The surgeons need to stretch the remaining stomach tissue and connect it with the esophagus. Measuring the oxygen saturation at this moment is crucial, and thus the device has to operate under spot-check or continuous run time to provide oxygen saturation measurement on the local tissue. To test the device for consistency and to estimate average battery life, the prototype was used continuously for a period of eight hours on two new AAA batteries without any change in accuracy. This far exceeds the required 45 minute run time requested by the sponsors. The oxygen saturation readings will be taken throughout the 8 hour period to check for variation in reading. Moreover, the Nonin sensor has an integrated power shut off feature that conserves battery when the LEDs and detector are not in use. The microcontroller also provides features for reducing battery consumption that are already implemented.

### Sterilization & Reusability

Device Casing: The existing oximeter is made using a styrene resin (ABSP-400) that can be used for animal testing, but not for the final prototype as this material is prone to cracking under misuse. The final device will be manufactured using high-performance medical grade polypropylene which is currently being used by companies such as ExxonMobil and Johnson & Johnson for medical devices. This polymer has excellent material properties that makes it suitable for intra-operative use and can be subjected to all types of sterilization techniques that are approved by the FDA. To increase the reusability of the device in the Operating Room, a disposable transparent polypropylene film will be used to cover the device during use on the stomach tissue. This ensures that constant sterilization of the entire device is not required, and saves time and resources.
Sterilization Methods The FDA requires all medical devices that will come into contact with body fluids be sterilizable using any of the following techniques:
Autoclaving: Proper autoclave treatment will inactivate all fungi, bacteria, viruses and also bacterial spores, which can be quite resistant. It will not necessarily eliminate all prions (proteinaceous & infectious). Although this method is the most common sterilization technique applied in hospitals and clinics, subjecting the device to extreme temperatures might affect the electronic components stored within the sensor head. Device performance testing at temperatures above 120° C. has not been conducted at the moment.
Ethylene Oxide Gas: Ethylene oxide gas kills bacteria (and their endospores), mold, and fungi, and can therefore be used to sterilize substances that would be damaged by sterilizing techniques such as pasteurization that rely on heat. Additionally, ethylene oxide is widely used to sterilize medical supplies such as bandages, sutures, and surgical implements. The overwhelming majority of medical items are sterilized with ethylene oxide. Preferred methods have been the traditional chamber sterilization method, where a chamber is flooded with a mix of ethylene oxide and other gases which are later aerated, and the more recent gas diffusion method developed in 1967 which relies on a bag that wraps the elements to be sterilized and acts as a mini-chamber in order to minimize gas consumption and make the process economically feasible for small loads. Other names for this alternative method for small loads are: Anprolene method, bag sterilization method or micro-dose sterilization method.

Irradiation (Electron beam, X-Rays, Gamma rays): If administered at appropriate levels, all of these forms of radiation can be used to sterilize objects, a technique used in the production of medical instruments and disposables, such as syringes as well as in the disinfestations and sterilization of food. Small doses of ionizing radiation (electron beam processing, X-rays and gamma rays) may be used to kill bacteria in food, or other organic material, including blood. Irradiation also includes (by the principle) microwave heating.

Liquid Chemical Agents: Using liquid chemicals that are not just disinfectants, but are classified as sterilants have advantages but also are limited due to the nature of reactions they have with medical grade plastics. Not only do the agents have to have a high efficacy (should be virucidal, bactericidal, tuberculocidal, fungicidal and sporicidal), but also must be non-toxic, non-staining, easy to use, easily disposable, have a long shelf-life and be reusable, and finally be cost effective. It also should have material compatibility and should produce negligible changes in either the appearance or function (especially optical clarity) of processed items, even after repeated cycling. It should not corrode instrument or cause deterioration of rubber, plastics, metals or other construction materials such as elastomers. Some common agents currently used are chlorine bleach, glutaraldehyde, formaldehyde, hydrogen peroxide, and peracetic acid.

### Power Dissipation Testing—Sensor Elements (IR/RED LED)

The power consumed by the electronic components in the device has to be minimal to ensure less heat generation and extend battery life. However, the sensing elements in the oximeter (Infra-Red and Red LED) have the highest consumption of power, and they also emit heat through photons into the tissue itself. Depending upon exposure time and the intensity of the lights used, this heat absorption into the tissue can cause significant damage. In a reported case, a patient was undergoing maxillofacial and nasal surgery for a period of 7 hours. A finger pulse oximeter was kept attached to the patient throughout the duration of the surgery. At the end, it was seen that combined with the positioning of the LED, the clip, and the intensity of light used, the finger experienced mild burns with deep circular burn marks indicative of the sensor diode (Baruchin A. M., et al., 1993). To eliminate such thermal burns occurring on more sensitive tissue surfaces such as those on the stomach and gastrointestinal tract, the maximum power dissipated in to the tissue must not exceed 30 mW/cm. The Nonin 8000-R sensor has a combined maximum average power dissipation of 2 mW (Red—0.8 mW, Infra-Red—1.2 mW, Nonin OEM III Specification & Technical Information).

### Device Accuracy Testing

Determining Accuracy

The above figure provides data for blood oxygen saturation measurements taken over ten independent trials. The three labeled traces correspond to measurements taken from our design prototype, an Onyx Finger Oximeter, and an SPO Medical Finger Oximeter. The two commercially available pulse oximeters were used as our gold standard during accuracy determinations.

### Accuracy Relative to Commercial Devices

For each trial, an average was taken for the two commercial oximeters. The difference between oxygen saturation for our prototype and the commercial devices□ average measurement was converted to a percentage. The final accuracy values for all the trials were averaged to produce our final relative accuracy of 3%.

Total Accuracy of Prototype

Due to the inherent +/−2% accuracy for each commercial oximeter, our total accuracy was adjusted to be 5%.

### Other Embodiments

### Wireless Data Transfer Via Bluetooth Technology

Signal output through Bluetooth to integrate with hospital/consumer display systems is one modification which will offer significant device improvement without requiring many modifications to be made to the existing prototype. This wireless module can be readily implemented to replace the serial cable currently used by the device.

### Pressure Application Monitoring Module

A small sensor may be implemented directly behind the sensor inside of the device to measure the application pressure induced by the user. The component may output an electrical warning signal (in the form of a light of buzzing sound) to warn the user of the device is being applied outside of the recommended pressure range. A warning signal will be produced if the applied pressure is greater than 40 kPa. One possible device to accomplish real-time pressure monitoring is a thin-beam load cell.

### Contact Application Monitoring Module

A surface contact sensor may be implemented on the distal surface of the device. The sensor may monitor contact with tissue by measuring the resistance between two electrodes. This is accomplished by passing a small amount of current or voltage across the electrodes. More than two electrodes may be used and controlled by the microcontroller to provide more information on the nature of contact.

### Temperature Application Monitoring Module

A thermistor or thermocouple may be implemented on the distal surface of the device or next to the primary optical sensors. Temperature is transduced by these devices as resistance changes which are detected electrically by the microcontroller and associated circuit. The potions and material around the temperature sensor may be adjusted as necessary.

### Compression Application Monitoring Module

A compression sensor including a strain-gauge may be mounted on any compressible component of the device include the head, the cuff, or the cover to provide an direct measure of the degree of motion and compression. Multiple sensors can be used to detected compression of multiple components and/or compression of a single component in multiple directions.

### Using Relative Measures

The operator may make a relative measurement in one region or using a standard and then press “set baseline” on the device to set the current measurement amplitude as the relative standard. At this point, subsequent measurements are displayed relative to this “pre-set baseline”. In another version, the user may use multiple regions or baseline to set “multiple set-points” in a manner analogous to a multi-point calibration. In another version, the user selects relative values from a pre-existing library generated by the same user or a different user.

### Developing the Sensor of the Device

One of the more ambitious modifications that can be made to our device would be to develop the sensor itself. There are several modifications that can be made in this area, including the geometrical arrangement of the sensor, using multiple photodetectors, using multiple LEDs, and using different optically isolating material. This would allow a sensor to be made that would be optimized specifically for its intended intraoperative use. Ideally, it would be tested under the appropriate pH, temperature, and physiological conditions. Further animal testing may also be considered.

With the future modifications taken into consideration, the proposed intraoperative pulse oximeter prototype has the potential to be implemented into a clinical patient-care setting. With wireless options and an easily adaptable graphical user interface, the device will take advantage of available technological options in the future of healthcare delivery. It shows great promise for its intraoperative application, and it is our hope that its development will be continued.

Based upon the foregoing it will be appreciated by those skilled in the art that we disclose intraoperative pulse oximeter technology to quantitatively assess oxygen saturation at a local site continuously and non-invasively on gastro-intestinal tissue during anastomosis in bowel resection surgery. The innovation enables the surgeon to attain better results during small bowel resection by providing real-time information of localized blood oxygen saturation, and based on the results, the surgeon performs an anastomosis at a site with more favorable blood oxygen saturation as a preventative measure to postoperative anastomotic leakage.

1. A handheld apparatus for in vivo examination of the viability of a biological tissue, comprising:
a sensor assembly comprising at least one optical emitter and at least one optical detector;
an optically opaque holder having an opening and containing at least the optical detector;
a shaft comprising a distal portion nearer to the holder and a proximal portion further from the holder;
a device head comprising a processor which is in communication with the sensor assembly through the shaft to provide data on the viability of the biological tissue; and
a power unit to power the optical emitter, optical detector and processor,
wherein at least the holder and the distal portion of the shaft are connected by a joint member.

2. The handheld apparatus of claim 1 further comprising a monitor in communication with the processor to display the data

3. The handheld apparatus of claim 2 wherein the data is displayed visually or by sound.

4. The handheld apparatus of claim 2, wherein the monitor is attached to the device head.

5. The handheld apparatus of claim 1, wherein the monitor is separate from the apparatus and in wireless or wired communication with the processor.

6. The handheld apparatus of claim 1, wherein the viability of the tissue is selected from the group consisting of oxygen level, carbon dioxide level, carbon monoxide level, temperature, metabolite level, average blood flow, uniformity of blood flow, density, pulse rate, tissue type, cell type, cell structure, cell death, ion concentration, hemorrhage, neuro-modulator concentration, and neurotransmitter concentration.

7. The handheld apparatus of claim 1, wherein the joint member is a universal or ball joint.

8. The handheld apparatus of claim 1, wherein the device head and the proximal portion of the shaft are connected by a joint member.

9. The handheld apparatus of claim 8, wherein the joint member is a universal or ball joint.

10. The handheld apparatus of claim 1, wherein the holder is capable of pivoting relative to the distal portion of the shaft to adjust the angle of the holder with the surface of the biological tissue.

11. The handheld apparatus of claim 1, wherein the holder comprises a cuff around the opening to at least partially block the ambient light from the optical sensor.

12. The handheld apparatus of claim 11, wherein the cuff is compliant.

13. The handheld apparatus of claim 11, wherein the cuff comprises a foam, a gel, rubber or spring.

14. The handheld apparatus of claim 1, wherein at least the opening of the holder is covered by a casing to prevent contact of at least the optical detector with the biological tissue, wherein the casing is substantially transparent to the light from the biological tissue.

15. The handheld apparatus of claim 14, wherein the casing is substantially transparent to the light emitted by the optical emitter.

16. The handheld apparatus of claim 2, wherein the monitor displays a measure of the viability of the biological tissue relative to another part of the biological tissue, another biological tissue, a predetermined value or a reference standard.

17. The handheld apparatus of claim 16, wherein the measure is displayed as a color, dial or bar scale.

18. The handheld apparatus of claim 1 further comprising a secondary sensor capable of detecting the position of the holder relative to the tissue, the position of the optical emitter relative to the tissue, the amount of ambient light the optical detector or the biological tissue is exposed to, or a characteristic of the tissue or the apparatus.

19. The handheld apparatus of claim 18, wherein the secondary sensor is capable of detecting the position of the apparatus relative to the tissue or the configuration of the apparatus.

20. The handheld apparatus of claim 19, wherein the secondary sensor is capable of detecting the angle between the holder and the distal portion of the shaft.

21. The handheld apparatus of claim 19, wherein the secondary sensor is capable of detecting the compression of the biological tissue by the holder.

22. The handheld apparatus of claim 18, wherein the secondary detector is capable of detecting the position of the holder, the distance the holder is away from the surface of the tissue, the contact between a portion of the holder and the tissue, contact between a portion of the holder and fluid near the tissue, or the pressure between the holder and the surface of the tissue.

23. The handheld apparatus of claim 18, wherein the characteristic of the tissue is the temperature of the surface of the biological tissue, pulse rate, regularity of pulse rate, blood flow, changes in blood flow, uniformity of blood flow, disruption of blood flow, tissue oxygenation, spatial distortion, compression of blood vessels or tissue, damage of blood vessels or tissue, tissue strain, tissue stress, tissue bending, tissue deflection, tissue stretching, tissue hemorrhage, tissue compression, moisture, or density.

24. The handheld apparatus of claim 18, wherein the data from the secondary sensor is displayed on a monitor.

25. The handheld apparatus of claim 1, wherein the holder further comprises a heating or cooling element or an element that delivers or removes fluid.
